(19) United States
(12) Patent Application Publication
(10) Pub. No.: US 2006/0177521 A1
(43) Pub. Date: Aug. 10, 2006

(54) HUMIDIFER SANITIZATION

(76) Inventors: Steven E. Bromberg, Livermore, CA (US); Elizabeth Crane, Martinez, CA (US); Scott Cumberland, Tracy, CA (US); Daniel Aaron Huitt, Esparto, CA (US); Diane Mellett, San Francisco, CA (US); William L. Smith, Pleasanton, CA (US)

Correspondence Address: THE CLOROX COMPANY, P.O. BOX 24305, OAKLAND, CA, 94623-1305, US

(21) Appl. No.: 11/379,467

(22) Filed: Apr. 20, 2006

Related U.S. Application Data
(63) Continuation-in-part of application No. 10/828,571, filed on Apr. 20, 2004.
Application No. 10/828,571 is a continuation-in-part of application No. 10/806,522, filed on Mar. 23, 2004.

(51) Int. Cl. A61K 33/14

(52) U.S. Cl. 424/661; 424/40

(57) ABSTRACT

A humidifier that delivers dilute hypohalous acid can effectively treat microganisms in the room while at the same time raising the humidity level. This humidifier system can be used to treat mold, microorganisms, and allergens, while being safe to use around people, pets, and food.

### CROSS REFERENCE TO RELATED APPLICATIONS

The present application is a continuation-in-part of Co-pending application Ser. No. No. 10/828,571 published as U.S. Pat. App. 2005/0216,291, which was filed Apr. 20, 2004, entitled “Method for Diluting Hypochlorite”, which is a continuation-in-part of Co-pending application Ser. No. 10/806,522 published as U.S. Pat. App. 2005/0214386, which was filed Mar. 23, 2004, entitled “Methods for Deactivating Allergens and Preventing Disease”, all of which is incorporated herein.

### BACKGROUND OF THE INVENTION

1. Field of the Invention

This invention relates to methods for delivering dilute hypohalous acid, hypohalous acid salt, and compositions containing these actives into the air using a humidifier. The invention also describes humidifiers, which deliver dilute hypohalous acid, hypohalous acid salt, and compositions containing these actives into the air. The method and devices are useful for treating allergen containing surfaces, hard surfaces, food contact surfaces, hospital surfaces, food surfaces, kitchen surfaces, bathroom surfaces, human surfaces, animal surfaces, military equipment, transportation equipment, children's items, plant surfaces, seeds, outdoor surfaces, soft surfaces, air, wounds, and medical instruments.

2. Description of the Related Art

Maintaining the proper indoor humidity level is extremely important. A humidity level that is too low can make breathing difficult. Very low humidity also increases static electricity, which causes allergens to remain airborne. This aggravates allergy symptoms and asthma. A humidity level that is too high provides an ideal environment for dust mites, mold, and mildew to thrive and grow. Operating a humidifier in the home can lead mold growth in the humidifier itself; mold growth that will be spread efficiently by the heating/cooling system to mold cross-contaminate the entire house.

U.S. Pat. No. 5,433,923 to Wolverton et al. describes the use of living plants for treating airborne spores, bacterial contamination, mold, fungi, and other microorganisms.

U.S. Pat. App. 2004/0214495 to Foss et al. describes antimicrobial fibers used in humidifiers to reduce or prevent the growth of organisms and prevent odor in humidifier systems. U.S. Pat. App. 2005/0025738 to Hall describes a humidifier solution that releases fragrance through the action of the humidifier, while providing a disinfectant to treat mold growth in the humidifier.

PCT App. WO2004/045654 to Hamilton et al. describes the use of chlorine dioxide or ethylene gas to remediate mold in a bathroom, kitchen, restaurant, gym, medical facility, locker room, or aquatic facility. The method requires sealing off a room prior to exposing the mold to the gas by dispersing the gas in a gas dispersion device, such as a flogger, a spray bottle, an atomizer, or a humidifier.

U.S. Pat. No. 6,033,704 to Talley describes the use of a gas stream containing an oxidant, such as hypochlorite, generated by an atomizer to preserve fruits and vegetables.

These technologies point out the difficulty in keeping indoor air both safe and healthy. Based on the prior art examples, the need exists for a simple technology that can be used to humidify indoor while limiting indoor contaminants.

### SUMMARY OF THE INVENTION

In accordance with the above objects and those that will be mentioned and will become apparent below, one aspect of the present invention is a method for treating a room comprising:
a. generating a dilute hypohalous acid vapor using a humidifier;
b. wherein the method is safe in the presence of a member of the group consisting of people, pets, food, or combinations thereof.

In accordance with the above objects and those that will be mentioned and will become apparent below, another aspect of the present invention is a humidifier comprising:
a. a water storage container comprising a dilute solution of hypohalous acid; and
b. a water vapor generating means;
c. wherein the humidifier generates water vapor comprising hypohalous acid.

In accordance with the above objects and those that will be mentioned and will become apparent below, another aspect of the present invention is a humidifier comprising:
a. a water storage container comprising a dilute solution of hypohalous acid; and
b. a water vapor generating means;
c. wherein the humidifier generates water vapor comprising hypohalous acid.

In accordance with the above objects and those that will be mentioned and will become apparent below, another aspect of the present invention is a device comprising:
a. a composition comprising hypohalous acid; and
b. a means to generate an aqueous vapor comprising hypohalous acid.

Further features and advantages of the present invention will become apparent to those of ordinary skill in the art in view of the detailed description of preferred embodiments below.

### DETAILED DESCRIPTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

As used herein and in the claims, the term “comprising” is inclusive or open-ended and does not exclude additional unrecited elements, compositional components, or method steps. Accordingly, the term “comprising” encompasses the more restrictive terms “consisting essentially of” and “consisting of”.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active) of the cleaning composition alone.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, nonionic, cationic and/or amphoteric agents.

Hypohalous Acid and Salts

Suitable hypohalous acids and salts may be provided by a variety of sources, including compositions that lead to the formation of positive halide ions and/or hypohalite ions; hypohalous acid, hypohalous acid salt, hypohalous acid generating species, hypohalous acid salt generating species; as well as compositions that are organic based sources of halides, such as chloroisocyanurates, haloamines, haloimines, haloimides and haloamides, or mixtures thereof. These compositions may also produce hypohalous acid or hypohalite species in situ. Suitable hypohalous acids and salts for use herein include the alkali metal and alkaline earth metal hypochlorites, hypobromites, hypoiodites, chlorinated trisodium phosphate dodecahydrates, potassium and sodium dichloroisocyanurates, potassium and sodium trichlorocyanurates, N-chloroimides, N-chloroamides, N-chlorosulfamide, N-chloroamines, chlorohydantoins such as dichlorodimethyl hydantoin and chlorobromo dimethylhydantoin, bromo-compounds corresponding to the chloro-compounds above, and compositions which generate the corresponding hypohalous acids, or mixtures thereof.

In one embodiment wherein the compositions herein are liquid, said hypohalite composition comprises an alkali metal and/or alkaline earth metal hypochlorite, or mixtures thereof. Compositions may comprise an alkali metal and/or alkaline earth metal hypochlorite selected from the group consisting of sodium hypochlorite, potassium hypochlorite, magnesium hypochlorite, lithium hypochlorite and calcium hypochlorite, and mixtures thereof.

The hypohalous acids and salt composition may be an equilibrium mixture of hypochlorous acid and hypochlorite salt, for example, sodium hypochlorite. The active species is present in an amount from above zero to about 15 weight percent of the composition, or from about 0.001 weight percent (10 ppm) to about 10 weight percent of the composition, or from about 0.005 (50 ppm) to about 5 weight percent of the composition. Dilute solutions of hypohalous acid and salt are generally below 1000 ppm.

The amount of available halogen oxidant in the composition is determined by placing samples of the composition into about 50 milliliters of distilled water, followed by addition of about 10 milliliters of a 10 weight/weight percent solution of potassium iodide and addition of about 10 milliliters of a 10 volume percent solution of sulfuric acid, the resulting mixture being well stirred. The resulting yellow to brown solution, whose color is the result of oxidation of free iodine ion (I⁻) to molecular iodine (I₂), was then volumetrically titrated to an essentially colorless endpoint by addition of standardized 0.1 Molar sodium thiosulfate (Na₂S₂O₃) titrant. Calculation then expresses the result as percent of available molecular chlorine (Cl₂), that is to say assigning two equivalents per mole of titrated hypohalite oxidant. Stability results are then expressed by repeated assays over time using identically prepared samples resulting from the same composition, normalized to 100 percent representative of the starting available chlorine measured initially.

During the course of evaluating various oxidants and antimicrobials for their allergen deactivating ability, we have found that a very dilute solution (on the order of 40-80 ppm) of primarily hypochlorous acid can effectively deactivate allergens. Presumably the low levels of oxidant are still able to break up the allergen proteins, rendering them biologically inert.

While still extremely effective, the low concentration and nearly neutral pH (6.9) of hypochlorous virtually eliminates surface damage. There is no sticky residue that can affect the feel of fabrics and there may be minimal dye damage. The solution may be aerosolized to treat air directly, or applied to surfaces.

Aerosols are known to have a low collision rate between denaturant and allergen particles. As a result, the denaturant must be used in high concentrations to be effective. Using this approach with conventional denaturants, which may be irritating or fragranced at high levels, can cause health problems. The use of a humidifier to deliver dilute hypohalous acid may reduce these problems.

Although hypohalous acid and hypohalous acid salt compositions can be useful over the entire pH range of 2 to 13, some benefits, such as the mold control, may require pH less than about pH 9, or less than pH 8, or less than pH 7. The compositions can include buffer systems, such as carboxylic acids and their salts, for example acetic acid or succinic acid. Other useful buffer systems would include borates, bicarbonates, hydrogen phosphates, and mixed metal silicates.

Dilute hypohalous acid and salt technology is described in U.S. Pat. App. 2005/0214,386, U.S. Pat. App. 2005/0216,291, U.S. Pat. App. 2005/0232,847, U.S. Pat. App. 2005/0232,848, U.S. Pat. App. 2005/0221,113, U.S. Pat. App. 2005/0233,900 and U.S. patent application Ser. No. 11/277,642 entitled “Antimicrobial Product Combination”, all of which are incorporated by reference herein.

Additional Actives

Additional actives that can be delivered with the humidifier include, for example, a perfume, a fragrance, an insect repellent, a fumigant, a disinfectant, a bactericide, an insecticide, a pesticide, a germicide, an acaricide, a sterilizer, a deodorizer, a fogging agent and mixtures of these. These actives can be delivered with the dilute hypohalous acid, in a separate vapor stream, in a mixed vapor stream, or as alternating vapors. Suitable fragrances for delivery are described in U.S. Pat. App. 2003/0024997 to Welch et al., which is incorporated herein.

Fragrances, or other incompatible actives that are sensitive to oxidizing solutions can be added and dispersed into the atmosphere by using individual, replaceable cartridges that liberate the fragrance when heated. Other incompatible actives can be delivered in the same way.

Humidifiers

Humidifiers deliver moisture into indoor spaces. One type of humidifier is an ultrasonic humidifier. Ultrasonic humidifiers generally comprise a container filled with water, which is excited by a piezoelectric disc that vibrates at a high frequency and in turn causes a phase change in the water by means of cavitation. An air stream directed onto the water surface carries the mist into the room to be humidified. The major drawback of both porous medium humidifiers and ultrasonic humidifiers is that the water staying in the container is not heated to its boiling point as in the steam generator and, is therefore susceptible to the growth of microorganisms, which are subsequently carried by the air stream into the room where it may be ingested by people. By delivering moisture from a sanitizing solution, this drawback can be avoided.

Another type of humidifier is a warm-air humidifier. Warm-air humidifiers share the benefits of steam generators in that growth of microorganisms is forestalled by heating the water to its boiling point. Also, warm-air humidifiers avoid the drawback of hot steam entering the room, since in this type of humidifier the steam is carried into the room as a mist mixed with air, at a temperature to be selected by judiciously choosing the ratio of steam and air. A typical warm-air humidifier is described in U.S. Pat. No. 4,564,746. This humidifier includes a heated evaporation chamber, which is enclosed to prevent leakage or damage and a fan adapted for dispersing the generated steam into the room via a cabinet passageway. The evaporation chamber is mounted on tracks, which permits it to be slid out of its enclosure for cleaning and servicing. The heating element, which is operationally enclosed in the chamber, is attached to a cover, which is likewise movable out of the humidifier cabinet for cleaning and servicing. PCT App. WO9514190 describes a portable and personal-sized electric warm air humidifier.

The humidifier can have replaceable or disposable cartridges containing dilute hypohalous acid that are readily placed in the humidifier. The replaceable cartridges can also be generators of hypohalous acid. The replaceable cartridges can also deliver additional ingredients. The humidifier can contain a fan. The materials used to manufacture the water container and transducer housing are compatible with the hypohalous acid solution to allow an effective treatment of microorganisms. In one embodiment of the application, the humidifier generates a dilute hypohalous acid vapor, which includes but is not limited to mists, aerosols, and gas. The hypohalous acid can prevent musty odor that emanates from the humidifier, which may be caused by mold in the humidifier vapor. The hypohalous acid can be used in the treatment of mold, treatment of allergens, treatment of bacteria, treatment of viruses, and combinations thereof.

Plug-In Air Treatment with Optional Fan

Plug-in diffusers are described in U.S. Pat. Nos. 4,849,606, and 5,937,140, both of which are incorporated herein by reference. A plug-in device can be designed to continuously or periodically release a fine mist of dilute hypochlorite. The plug-in can also optionally contain a fan or additionally release a fragrance. The device can kill germs and remove allergens while being safe to use around kids, pets, and food.

Self-Generating Steam Apparatus

The device can be a self-generating steam apparatus as described in U.S. Pat. 2005/0262757 to Wong et al. that contains a self-steaming (including, vaporizing) composition such that the vaporizer is portable, has its own energy source, and is not dependent upon an external source of energy for operation. In one embodiment, a sub-article comprising the composition is contained within the vaporizer article, such that upon activation the composition is self-steaming (including, self-vaporizing) for the benefit of the user. In one embodiment, the composition interacts with air to generate heat and water vapor containing dilute hypohalous acid. For example, the composition may be activated as follows: The article comprising the composition may include an oxygen impermeable plastic overwrap. A tear-tab or notch may be included on the overwrap for easy access by a user. Instructions may be included with the enclosure instructing a user to tear open the overwrap to remove the article comprising the self-steaming composition. This opening action immediately mixes oxygen contained in the ambient air with the composition to initiate the self-steaming process.

Portable Devices

The device can contain an energy source, such as batteries, and can also contain a means for allowing recharging of rechargeable internal batteries via such means as a plug or port such that the consumer can conveniently recharge the batteries. Other means of providing energy sources that allow the device to be portable include methanol fuel cells or minerals that generate heat upon mixture with water, for example, mixing water with anhydrous calcium oxide. Portable devices would allow for disposable humidifiers that could be taken for on the go occasions. For example, such systems could fit in the cup holders of vehicles.

Electrolytically Generated Hypohalous Acid

The device may be a self-generating plug-in or portable device, for example as described in U.S. Pat. App. 2003/0213704 to Scheper et al and U.S. Pat. App. 2005/0067300 to Tremblay. The device may contain an electrochemical cell to generate dilute hypohalous acid and a mechanism to evaporate the hypohalous acid solution into the air. The electrochemical cells and/or electrolytic devices are those cells and/or devices that are self-powered and self- contained and which draw their electrical power from the unattached electrolytic device itself and/or alternatively from a building's electrical power supply to produce electrolyzed water. The device can be plugged in or can contain power to supply for the electrochemical cell, the power for any pumping means, the power for any propulsion means, the power for any indication or control means, and the like. The devices can comprise a housing that can be sealed or can be sealable to prevent electrolytic solution from entering the housing, except as intended. The body can have an inlet port, through which electrolytic solution can pass through to the electrochemical cell, contained therein.

Full Room Treatment and Personal Devices

An aerosol device can be placed in the center of a room, then the aerosol device is activated and in a few minutes the entire contents are expelled and the air and surfaces of the room to kill germs and remove allergens. The aerosol device can be safe to use around kids, pets, and food. As an alternative to the aerosol device, a canister containing the active with a fan or a canister with a heat generating mechanism to deliver the active. This technology can also be used to deliver dilute hypohalous acid to a person. Suitable personal devices to deliver actives for respiratory treatment are disclosed in PCT App. WO0162264 to Zawadzki et al., which describes suitable dispensers including self-milling dry powder dispensers for actives as described in U.S. App. 2005/0233900. These personal device can be use to deliver dilute hypohalous acid in a liquid nebulisers or dry powders containing hypohalous acid.

Santizing Tablet

A tablet can dissolve in water to deliver low levels of hypohalous acid at neutral to acidic pH. The tablet may effervesce. The tablet can be used after the kids take a bath by tossing the tablet in a full tub before draining and the tub and bath toys will be sanitized. The tablet can also be used to sanitize the kitchen sink and cutting board, used in a humidifier, washing machine, and dishwasher. The tablet is safe to use around kids, pets, and food.

### EXAMPLES

Effect of Dilute Bleach on Humidifier

In separate experiments, dilute hypochlorite and water were placed in a humidifier in an enclosed 6 by 6 by 6 ft room. Petri dishes containing TSA agar inoculated with S. aureus were placed 30 inches and 60 inches from the humidifier. The humidifier was run for 1.5 hour. A 2 to 5 log reduction was observed on incubated plates placed in the room with dilute hypohalous acid compared to the water control.

Two different humidifiers were used, a Reli-on Ultrasonic Humidifier Model H-0565-0 with nickel transducer and a Fujitronic Ultrasonic Humidifier Model FB-602 with titanium transducer. As shown in Table 1 below, the Reli-on Humidifier caused a significant drop in the pH of the hypochlorite solution, indicating possible interaction with the nickel transducer and/or the materials that comprised the water container and transducer housing.

TABLE 1

| | Humidifier | | | |
|---|---|---|---|---|
| | Reli-on | | Fujitronic | |
| Run Time | Initial | 1.5 Hours | Initial | 1.5 Hours |
| Weight if Solution in | 1000 | 410 | 1000 | 465 |
| humidifer (g) | | | | |
| Hypochlorite | 123 | 82 | 123 | 79 |
| concentration (ppm) | | | | |
| pH | 5.52 | 4.77 | 5.52 | 5.42 |

Effect of Concentration and pH on Safety

An ultrasonic humidifier was run with bleach diluted to moderate concentration with deionized water and high pH and with low concentration and neutral pH. Black cloth was placed under the humidifier to measure dye damage. The moderate concentration bleach had extensive dye damage, while the low concentration bleach had none, as shown in Table 2.

TABLE 2

Humidifier
Kaz Ultrasonic
Humidifier, 5.5 hours

| | | |
|---|---|---|
| Hypochlorite | 3759 | 78 |
| concentration (ppm) | | |
| pH | 10.7 | 7.35 |
| Dye damage | Yes | No |

While various patents have been incorporated herein by reference, to the extent there is any inconsistency between incorporated material and that of the written specification, the written specification shall control. In addition, while the invention has been described in detail with respect to specific embodiments thereof, it will be apparent to those skilled in the art that various alterations, modifications and other changes may be made to the invention without departing from the spirit and scope of the present invention. It is therefore intended that the claims cover all such modifications, alterations and other changes encompassed by the appended claims.

We claim:

1. A method for treating a room comprising:
a. generating a dilute hypohalous acid vapor using a humidifier;
b. wherein the method is safe in the presence of a member of the group consisting of people, pets, food, or combinations thereof.

2. The method of claim 1, wherein the method is safe in the presence of people.

3. The method of claim 1, wherein the humidifier is an ultrasonic humidifier.

4. The method of claim 1, wherein the ultrasonic humidifier comprises a titanium transducer.

5. The method of claim 1, wherein the humidifier comprises an aqueous solution comprising less than 1000 ppm hypohalous acid and salt of hypohalous acid.

6. The method of claim 5, wherein the aqueous solution has a pH less than 10.

7. The method of claim 5, wherein the aqueous solution has a pH less than 8.

8. The method of claim 1, wherein the humidifier contains a hypohalous acid generator.

9. The method of claim 1, wherein the humidifier is portable.

10. The method of claim 1, wherein the humidifier comprises a fan.

11. The method of claim 1, wherein the humidifier comprises an energy source.

12. The method of claim 1, wherein the method is used for a treatment selected from the group consisting of treatment of mold, treatment of allergens, treatment of bacteria, treatment of viruses, and combinations thereof.

13. The method of claim 12, wherein the method is used for the treatment of mold.

14. The method of claim 12, wherein the method is used for the treatment of allergens.

15. The method of claim 12, wherein the method is used for the treatment of bacteria.

16. The method of claim 12, wherein the method is used for the treatment of viruses.

17. The method of claim 12, wherein the humidifier delivers an additional benefit.

18. The method of claim 17, wherein the additional benefit is fragrance delivery.

19. A humidifier comprising:
a. a water storage container comprising a dilute solution of hypohalous acid; and
b. a water vapor generating means;
c. wherein the humidifier generates water vapor comprising hypohalous acid.

20. The humidifier of claim 19; wherein the water storage container is a replaceable cartridge.

21. A humidifier comprising:
a. a hypohalous acid generating means; and
b. a water vapor generating means;
c. wherein the humidifier generates water vapor comprising hypohalous acid.

22. A device comprising:
a. a composition comprising hypohalous acid; and
b. a means to generate an aqueous vapor comprising hypohalous acid.

23. The device of claim 22, wherein the device is used to treat a person.
